(19) United States
(12) Patent Application Publication
(10) Pub. No.: US 2024/0216520 A1
(43) Pub. Date: Jul. 4, 2024

(54) PEPTIDE-SMALL INTERFERING RNA-HYALURONIC ACID NANOPARTICLES AND METHODS OF USE THEREOF

(71) Applicant: UNIVERSITY OF SOUTH FLORIDA, Tampa, FL (US)

(72) Inventors: Samuel WICKLINE, Tampa, FL (US); Hua PAN, Tampa, FL (US)

(21) Appl. No.: 18/554,285

(22) PCT Filed: Apr. 6, 2022
(86) PCT No.: PCT/US2022/023609

§ 371 Date: Oct. 6, 2023

Related U.S. Application Data
(60) Provisional application No. 63/171,176, filed on Apr. 6, 2021.

(51) Int. Cl. A61K 47/64; A61K 47/61

(52) U.S. Cl. CPC A61K 47/6455; A61K 47/61

(57) ABSTRACT

The present disclosure provides nanoparticle compositions and their use in the treatment of viral infections.

### CROSS-REFERENCE TO RELATED APPLICATIONS

This application claims the benefit of priority to U.S. Provisional Application No. 63/171,176, filed Apr. 6, 2021, the disclosure of which is incorporated herein by reference in its entirety.

### TECHNICAL FIELD

This disclosure relates to nanoparticle compositions and their use in the treatment of viral infections.

### BACKGROUND

Viral infections account for a very large fraction of infectious disease mortality and morbidity worldwide. The occurrence of many viral infections has been limited by the development of vaccines that prevent infections in many populations. Despite this, there are still many remaining viral pathogens for which vaccines have not been developed and for which further treatment options are limited. In fact, many viral infections are treated by either simply by allowing the infection to run its course or by the administration of palliative therapies. Many of the available therapeutics that directly treat the virus may also cause side effects due to systemic dosing. There is a clear need for new therapeutic platforms for the treatment of viral infections that are specific targeted to the viruses in addition to the virus-infected tissues.

### SUMMARY

The present disclosure provides compositions that may be used in the treatment of subjects with viral infections. The compositions comprise small interfering RNA (siRNA) that interferes with a viral ribonucleotide complexed with a peptide that allows endosomal release in a virus infected cell.

In one aspect, the present disclosure provides a composition comprising a peptide-siRNA complex, wherein the peptide-siRNA complex comprises: a peptide: a small interfering RNA (siRNA) that interferes with a viral ribonucleotide; and a hyaluronic acid (HA): wherein the peptide is non-lytic in circulation and capable of affecting release of the siRNA from an endosome of a virus-infected cell; and wherein the peptide comprises an amino acid sequence with at least 80% identity to the amino acid sequence of SEQ ID NO: 1 (VLTTGLPALISWIRRRHRRHC).

In some embodiments, the peptide-siRNA complex is about 10 nm to about 150 nm in average diameter, for example from about 10 to about 50 nm or from about 60 to about 100 nm in average diameter. In other embodiments, the peptide-siRNA complex is about 40 nm to about 80 nm in average diameter.

In some embodiments, the hyaluronic acid coats the peptide-siRNA complex. In other embodiments, the hyaluronic acid is integrated into the peptide-siRNA complex. In some embodiments, the hyaluronic acid comprises a hyaluronic acid conjugate. In some embodiments, the hyaluronic acid conjugate comprises hyaluronic acid covalently bound to a cell-targeting ligand. In some embodiments, the hyaluronic acid conjugate comprises hyaluronic acid covalently bound to an angiotensin-converting enzyme 2 (ACE2) protein ligand.

In some embodiments, the peptide comprises an amino acid sequence with at least 85% identity, at least 90% identity, or at least 95% identity to the amino acid sequence of SEQ ID NO: 1. In some embodiments, the peptide comprises SEQ ID NO: 1. In some embodiments, the peptide consists of an amino acid sequence with at least 85% identity, at least 90% identity, or at least 95% identity to the amino acid sequence of SEQ ID NO: 1. In some embodiments, the peptide consists of the amino acid sequence of SEQ ID NO: 1.

In some embodiments, the siRNA interferes with at least a portion of the RNA genome of severe acute respiratory syndrome coronavirus 2 (SARS-COV-2).

The present disclosure also provides a method of treating a viral infection in a subject in need thereof comprising administering to the subject a therapeutically effective amount of the composition as described herein. In some embodiments, the viral infection comprises SARS-COV-2.

The present disclosure further provides a method of delivering siRNA to a virus-infected cell comprising contacting the cell with the composition described herein. In some embodiments, the cell is infected with SARS-Cov-2.

The details of one or more embodiments of the disclosure are set forth in the description below. Other features, objects, and advantages of the disclosure will be apparent from the description and drawings, and from the claims.

### DETAILED DESCRIPTION

Reference will now be made in detail to the embodiments of the invention. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs.

The following definitions are provided for the full understanding of terms used in this specification.

### Definitions

As used herein, the article “a,” “an,” and “the” means “at least one,” unless the context in which the article is used clearly indicates otherwise.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed herein.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds. A polypeptide is comprised of approximately twenty, standard naturally occurring amino acids, although natural and synthetic amino acids which are not members of the standard twenty amino acids may also be used. The standard twenty amino acids include alanine (Ala, A), arginine (Arg. R), asparagine (Asn, N), aspartic acid (Asp. D), cysteine (Cys, C), glutamine (Gln, Q), glutamic acid (Glu, E), glycine (Gly, G), histidine, (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The terms “polypeptide sequence” or “amino acid sequence” are an alphabetical representation of a polypeptide molecule.

Conservative substitutions of amino acids in proteins and polypeptides are known in the art. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the polypeptides provided herein.

Substantial changes in protein function or immunological identity are made by selecting substitutions that are less conservative, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl: (b) a cysteine or proline is substituted for (or by) any other residue: (c) a residue having an electropositive side chain, e.g., lysyl, arginyl, or histidyl, is substituted for (or by) an electronegative residue, e.g., glutamyl or aspartyl: or (d) a residue having a bulky side chain, e.g., phenylalanine, is substituted for (or by) one not having a side chain, e.g., glycine, in this case, (e) by increasing the number of sites for sulfation and/or glycosylation.

A “variant” refers to a molecule substantially similar in structure. Thus, in one embodiment, a variant refers to a protein whose amino acid sequence is similar to a reference amino acid sequence, but does not have 100% identity with the respective reference sequence. The variant protein has an altered sequence in which one or more of the amino acids in the reference sequence is deleted or substituted, or one or more amino acids are inserted into the sequence of the reference amino acid sequence. As a result of the alterations, the variant protein has an amino acid sequence which is at least 60%, 70%, 75%, 80%, 85%, 90%, or 95% identical to the reference sequence. For example, variant sequences which are at least 95% identical have no more than 5 alterations, i.e. any combination of deletions, insertions or substitutions, per 100 amino acids of the reference sequence.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below; or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below; the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues: always >0)) and N (penalty score for mismatching residues: always <0)). For amino acid sequences, a scoring matrix is used to calculate the cumulative score. Extension of the word hits in each direction are halted when: the cumulative alignment score falls off by the quantity X from its maximum achieved value: the cumulative score goes to zero or below, due to the accumulation of one or more negative-scoring residue alignments: or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

As used herein, the phrase “functions substantially similar to a peptide comprising SEQ ID NO: X” refers to a substantially non-lytic in circulation and/or non-cytotoxic peptide that is capable of affecting the release of a polynucleotide from an endosome.

The term “non-lytic” means that the lipid bilayer surrounding a cell typically is not compromised upon contact with the peptide. The integrity of the lipid bilayer may be assessed by the improper entry or exit of cellular or extracellular components into a cell. For example, cellular proteins and/or organelles may leak out of a cell with a compromised lipid bilayer. Alternatively, extracellular components (i.e., those that normally do not enter via gap junctions, for example) may enter a cell with a compromised lipid bilayer. It should be noted, however, that the peptide may penetrate the lipid bilayer of a cell and enter the interior of the cell, but in doing so the integrity of the lipid bilayer is not affected.

The term “non-cytotoxic” indicates that the cell typically is not killed upon contact with the peptide.

As used herein, the term “coat” or “coating” may refer to the interaction of a nanoparticle (peptide-polynucleotide complex) with a compound through non-covalent bonds, or to the covalent bonding of a nanoparticle and a compound.

The term “about” as used herein when referring to a measurable value such as an amount, a percentage, and the like, is meant to encompass variations of ±20%, ±10%, ±5%, or ±1% from the measurable value.

### Peptide

In one aspect, a peptide-siRNA complex of the invention comprises a peptide. In general, a peptide of the invention is derived from melittin and modified to attenuate its cytotoxicity while maintaining its propensity for interacting with membrane bilayers. Further, the peptide is substantially non-lytic in circulation and non-cytotoxic to cells. Preferably, a peptide-siRNA complex of the invention comprises a peptide that (1) has a function substantially similar to a peptide with an amino acid sequence of SEQ ID NO: 1, or (2) has an amino acid sequence with similarity or identity to the amino acid sequence of SEQ ID NO: 1.

As used herein, the phrase “functions substantially similar to a peptide comprising SEQ ID NO: 1” refers to a substantially non-lytic in circulation and/or non-cytotoxic peptide that is capable of affecting the release of a polynucleotide from an endosome. In some embodiments, a peptide of the invention is non-lytic in circulation. The term “non-lytic” means that the lipid bilayer surrounding a cell typically is not compromised upon contact with the peptide. The integrity of the lipid bilayer may be assessed by the improper entry or exit of cellular or extracellular components into a cell. For example, cellular proteins and/or organelles may leak out of a cell with a compromised lipid bilayer. Alternatively, extracellular components (i.e., those that normally do not enter via gap junctions, for example) may enter a cell with a compromised lipid bilayer. It should be noted, however, that the peptide may penetrate the lipid bilayer of a cell and enter the interior of the cell, but in doing so the integrity of the lipid bilayer is not affected. In other embodiments, the peptide of the invention is substantially non-cytotoxic. The term “non-cytotoxic” indicates that the cell typically is not killed upon contact with the peptide. Typically, a peptide of the invention decreases cell viability by no more than about 10%, for example no more than about 7%, no more than about 5%, or no more than about 3%. In certain embodiments, a peptide of the invention is non-lytic in circulation and non-cytotoxic.

A peptide of the invention is capable of being associated with an siRNA as described herein. Thus, in one aspect, a peptide of the invention comprises at least one cationic region that interacts with an siRNA. Typically, a cationic region has 2 or more contiguous, basic amino acids. Importantly, a peptide of the invention also possesses an endosomolytic capacity, which allows it to affect the release of the siRNA from an endosome and into the cytoplasm of a cell. The term “endosomolytic” can be used to describe substances that initiate or facilitate the lysis of endosomes. At low pH in an endosome (for example, a pH less than about 6.2, less than about 5.5, or less than about 4.5), protonation of histidine residues of a peptide of the invention promotes disassembly of the peptide-siRNA complex, which releases the peptide to permeabilize the endosomal membrane for siRNA release. Thus, in another aspect, a peptide of the invention comprises one or more histidine residues located adjacent to or within at least one cationic region of the peptide. The endosomolytic capacity of a peptide of the invention obviates the need for additional endosomolytic agents, such as chloroquine, fusogenic peptides, inactivated adenoviruses and polyethyleneimine, for releasing the siRNA from endosomes for delivery into the cytoplasm of a cell. Such known endosomolytic agents have negative effects on cells and may increase cytotoxicity during transfection.

In some embodiments, a peptide of the invention comprises an amino acid sequence of SEQ ID NO: 1. In some embodiments, a peptide of the invention consists of an amino acid sequence of SEQ ID NO: 1. In some embodiments, a peptide of the invention comprises a variant of the amino acid sequence of SEQ ID NO: 1.

In some embodiments, a peptide of the invention comprises an amino acid sequence that has at least 80% identity to the amino acid sequence of SEQ ID NO: 1, wherein the peptide is non-lytic in circulation and is capable of affecting the release of the siRNA from an endosome of a cell. The peptide comprising an amino acid sequence that has at least 80% identity to the amino acid sequence of SEQ ID NO: 1, can have about 90%, about 85%, about 90%, about 95%, or more identity to the amino acid sequence of SEQ ID NO: 1. A peptide of the invention comprising an amino acid sequence that has at least 80% identity to the amino acid sequence of SEQ ID NO: 1 may comprise one or more amino acids that have been conservatively substituted as long as the resulting peptide functions substantially similar to a peptide comprising the amino acid sequence of SEQ ID NO: 1.

In another aspect, the present invention provides an amino acid sequence that has at least 80% identity to the amino acid sequence of SEQ ID NO: 1 and encodes a peptide that is non-lytic in circulation and capable of affecting the release of the siRNA from an endosome of a cell. In some embodiments, the amino acid sequence has at least 80% identity, at least 85% identity, at least 90% identity, or at least 95% identity to the amino acid sequence of SEQ ID NO: 1. In other embodiments, the amino acid sequence is SEQ ID NO. 1.

A peptide of the invention may be produced using a variety of techniques known in the art. The peptides may be isolated using standard techniques, may be synthesized using standard techniques, or may be purchased or obtained from a depository.

When a peptide of the invention contains a C-terminal thiol in the form of a cysteine residue, a peptide of the invention may be able to form a disulfide bond with another free thiol group, for example, with a free thiol group from the same or different peptide. A skilled artisan can readily determine whether dimer formation does or does not improve the delivery of the siRNA of the present invention. Dimerization may be induced by incubation of free peptide in 20% DMSO for 24-72 hours, or by other methods known in the art. As a non-limiting example, free thiols may be quantified by colorimetric assays using Ellman's reagent.

A peptide of the invention may be labeled. Non-limiting examples of suitable labels include fluorescent labels, chemiluminescent labels, radioactive labels, colorimetric labels, magnetic resonance labels, or other labels known to be detectable by standard imaging methods. Methods of labeling peptides are well known in the art.

Small Interfering RNA (siRNA)

In another aspect, the peptide-siRNA complex of the invention comprises a small interfering RNA (siRNA) which is capable of regulating or inhibiting the expression of a viral ribonucleotide expressed in a virus-infected cell. In general, an siRNA of the present disclosure is capable of disrupting expression of a viral ribonucleotide sequence expressed in a virus-infected cell. As used herein, “disrupting expression of a viral polynucleotide” may be used to describe any decrease in the expression level of a viral ribonucleotide, or a protein translated from the viral ribonucleotide, when compared to a level of expression of the viral ribonucleotide in a virus-infected cell that was not treated with a peptide-siRNA complex of the present invention.

In general, an siRNA comprises a double-stranded RNA molecular that ranges form about 15 to about 29 nucleotides in length. In some embodiments, the siRNA may be 16, 17, 18, 19, 20, 21, 22, 23, 24, 25, 26, 27, 28 or 29 nucleotides in length. In other embodiments, the siRNA may be about 16 to about 18, about 17 to about 19, about 21 to about 23, about 24 to about 27, or about 27 to about 29 nucleotides in length. An siRNA may optionally further comprise one or two single-stranded overhangs, e.g., a 5′ overhand on one or both ends, a 3′ overhand on one or both ends, or a combination thereof. The siRNA may be formed from two RNA molecules that hybridize together, or alternatively, may be generated from a short hairpin RNA (shRNA). In some embodiments, the two strands of the siRNA may be completely complementary, such that no mismatches or bulges exist in the duplex formed between the two sequences. In other embodiments, the two strands of the siRNA may be substantially complementary, such that one or more mismatches and/or bulges may exist in the duplex formed between the two sequences. In certain embodiments, one of both of the 5′ ends of the siRNA may have a phosphate group, while in other embodiments one or both of the 5′ ends lack a phosphate groups. In other embodiments, one or both of the 3′ ends of the siRNA may have a hydroxy group, while in other embodiments one or both of the 5′ ends lack a hydroxyl group.

One strand of the siRNA, which is referred to as the “antisense strand” or “guide strand”, includes a portion that hybridizes with a target transcript. A target transcript refers to a ribonucleotide sequence expressed by a cell for which it is desired expression be disrupted. In the context of a therapeutic composition of the invention, disrupting expression of a target transcript may produce a beneficial effect. In preferred embodiments, the antisense strand of the siRNA may be completely complementary with a region of the target transcript, i.e., it hybridized to the target transcript without a single mismatch or bulge over a target region between about 15 to about 29 nucleotides in length, preferably at least 16 nucleotides in length, and more preferably about 18-20 nucleotides in length. In other embodiments, the antisense strand may be substantially complementary to the target region, i.e., one or more mismatches and/or bulges may exist in the duplex formed by the antisense strand and the target transcript. Typically, siRNAs are targeted to exonic sequences of the target transcript. Those of skill in the art are familiar with programs, algorithms, and/or commercial services that design siRNAs for target transcripts. An example is the Rosetta siRNA Design Algorithm (Rosetta Inpharmatics, North Seattle, WA), MISSION® siRNA (Sigma-Aldrich St. Louis, MO) and siGENOME siRNA (Therma Scientific). The siRNA may be enzymatically synthesized in vitro using methods well known to those of skill in the art. Alternatively, the siRNA may be chemically synthetized using oligonucleotide synthesis techniques that are well known in the art.

Generally speaking, the promoters utilized to direct in vivo expression of the one or more siRNA transcription units may be promoters for RNA Polymerase III (Pol III). Certain Pol III promoters, such as U6 or H1 promoters, do not require cis-acting regulatory elements within the transcribed region, and thus are preferred in certain embodiments. In other embodiments, promoters for Pol II may be used to drive expression of the one or more siRNA transcription units. In some embodiments, tissue-specific, cell-specific, or inducible Pol II promoters may be used.

A construct that provides a template for the synthesis of siRNA may be produced using standard recombinant DNA methods and into any of a wide variety of different vectors suitable for expression in eukaryotic cells. Guidance may be found in Current Protocols in Molecular Biology (Ausubel et al., John Wile & Sons, New York, 2003) or Molecular Cloning: A Laboratory Manual (Sambrook & Russell, Cold Spring Harbor Press, Cold Spring Harbor, NY, 3rd Edition, 2001). Those of skill in the art also appreciate that vectors may comprise additional regulatory sequences (e.g., termination sequence, translational control sequence, etc.), as well as selectable marker sequences. DNA plasmids are known in the art, including those based on pBR322, PUC, and so forth. Since many expression vectors already contain a suitable promoter or promoters, it may only be necessary to insert the nucleic acid sequence that encodes the siRNA of interest at an appropriate location with respect to the promoter(s). Viral vectors may also be used to provide intracellular expression of siRNA agents. Suitable viral vectors include retroviral vectors, lentiviral vectors, adeno-associated virus vectors, herpes virus vectors, and so forth.

Nucleic acid sequences of the invention may be obtained using a variety of different techniques known in the art. The nucleotide sequences, as well as homologous sequences, may be isolated using standard techniques, purchased or obtained from a depository. Once the nucleotide sequence is obtained, it may be amplified for use in a variety of applications, using methods known in the art.

Exemplary viruses whose viral ribonucleotides may be inhibited by compositions described herein include viruses which belong to the following none exclusive list of families: Adenoviridae, Arenaviridae, Astroviridae, Baculoviridae, Barnaviridae, Betaherpesvirinae, Birnaviridae, Bromoviridae, Bunyaviridae, Caliciviridae, Chordopoxvirinae, Circoviridae, Comoviridae, Coronaviridae, Cystoviridae, Corticoviridae, Entomopoxvirinae, Filoviridae, Flaviviridae, Fuselloviridae, Geminiviridae, Hepadnaviridae, Herpesviridae, Gammaherpesvirinae, Inoviridae, Iridoviridae, Leviviridae, Lipothrixviridae, Microviridae, Myoviridae, Nodaviridae, Orthomyxoviridae, Papovaviridae, Paramyxoviridae, Paramyxovirinae, Partitiviridae, Parvoviridae, Phycodnaviridae, Picornaviridae, Plasmaviridae, Pneumovirinae, Podoviridae, Polydnaviridae, Potyviridae, Poxviridae, Reoviridae, Retroviridae, Rhabdoviridae, Sequiviridae, Siphoviridae, Tectiviridae, Tetraviridae, Togaviridae, Tombusviridae, and Totiviridae.

Specific examples of suitable viruses whose viral ribonucleotides may be inhibited by the compositions described herein include, but are not limited to, Mastadenovirus, Human adenovirus 2, Aviadenovirus, African swine fever virus, arenavirus, Lymphocytic choriomeningitis virus, Ippy virus, Lassa virus, Arterivirus, Human astrovirus 1, Nucleopolyhedrovirus, Autographa californica nucleopolyhedrovirus, Granulovirus, Plodia interpunctella granulovirus, Badnavirus, Commelina yellow mottle virus, Rice tungro bacilliform, Barnavirus, Mushroom bacilliform virus, Aquabirnavirus, Infectious pancreatic necrosis virus, Avibirnavirus, Infectious bursal disease virus, Entomobirnavirus, Drosophila X virus, Alfamovirus, Alfalfa mosaic virus, Ilarvirus, Ilarvirus Subgroups 1-10, Tobacco streak virus, Bromovirus, Brome mosaic virus, Cucumovirus, Cucumber mosaic virus, Bhanja virus Group, Kaisodi virus, Mapputta virus, Okola virus, Resistencia virus, Upolu virus, Yogue virus, Bunyavirus, Anopheles A virus, Anopheles B virus, Bakau virus, Bunyamwera virus, Bwamba virus, C virus, California encephalitis virus, Capim virus, Gamboa virus, Guama virus, Koongol virus, Minatitlan virus, Nyando virus, Olifantsvlei virus, Patois virus, Simbu virus, Tete virus, Turlock virus, Hantavirus, Hantaan virus, Nairovirus, Crimean-Congo hemorrhagic fever virus, Dera Ghazi Khan virus, Hughes virus, Nairobi sheep disease virus, Qalyub virus, Sakhalin virus, Thiafora virus, Crimean-congo hemorrhagic fever virus, Phlebovirus, Sandfly fever virus, Bujaru complex, Candiru complex, Chilibre complex, Frijoles complex, Punta Toro complex, Rift Valley fever complex, Salehabad complex, Sandfly fever Sicilian virus, Uukuniemi virus, Uukuniemi virus, Tospovirus, Tomato spotted wilt virus, Calicivirus, Vesicular exanthema of swine virus, Capillovirus, Apple stem grooving virus, Carlavirus, Carnation latent virus, Caulimovirus, Cauliflower mosaic virus, Circovirus, Chicken anemia virus, Closterovirus, Beet yellows virus, Comovirus, Cowpea mosaic virus, Fabavirus, Broad bean wilt virus 1, Nepovirus, Tobacco ringspot virus, Coronavirus, Avian infectious bronchitis virus, Bovine coronavirus, Canine coronavirus, Feline infectious peritonitis virus, Human coronavirus 299E, Human coronavirus OC43, Murine hepatitis virus, Porcine epidemic diarrhea virus, Porcine hemagglutinating encephalomyelitis virus, Porcine transmissible gastroenteritis virus, Rat coronavirus, Turkey coronavirus, Rabbit coronavirus, Torovirus, Berne virus, Breda virus, Corticovirus, Alteromonas phage PM2, Pseudomonas Phage phi6, Deltavirus, Hepatitis delta virus, Dianthovirus Carnation ringspot virus, Red clover necrotic mosaic virus, Sweet clover necrotic mosaic virus, Enamovirus, Pea enation mosaic virus, Filovirus, Marburg virus, Ebola virus Zaire, Flavivirus, Yellow fever virus, Tick-borne encephalitis virus, Rio Bravo Group, Japanese encephalitis, Tyuleniy Group, Ntaya Group, Uganda S Group, Dengue Group, Modoc Group, Pestivirus, Bovine diarrhea virus, Hepatitis C virus, Furovirus, Soil-borne wheat mosaic virus, Beet necrotic yellow vein virus, Fusellovirus, Sulfobolus virus 1, Subgroup I, II, and III geminivirus, Maize streak virus, Beet curly top virus, Bean golden mosaic virus, Orthohepadnavirus, Hepatitis B virus, Avihepadnavirus, Alphaherpesvirinae, Simplexvirus, Human herpesvirus 1, Varicellovirus, Human herpesvirus 3, Cytomegalovirus, Human herpesvirus 5, Muromegalovirus, Mouse cytomegalovirus 1, Roseolovirus, Human herpesvirus 6, Lymphocryptovirus, Human herpesvirus 4, Rhadinovirus, Ateline herpesvirus 2, Hordeivirus, Barley stripe mosaic virus, Hypoviridae, Hypovirus, Cryphonectria hypovirus 1-EP713, Idaeovirus, Raspberry bushy dwarf virus, Inovirus, Coliphage fd, Plectrovirus, Acholeplasma phage L51, Iridovirus, Chilo iridescent virus, Chloriridovirus, Mosquito iridescent virus, Ranavirus, Frog virus 3, Lymphocystivirus, Lymphocystis disease virus flounder isolate, Goldfish virus 1, Levivirus, Enterobacteria phage MS2, Allolevirus, Enterobacteria phage Qbeta, Lipothrixvirus, Thermoproteus virus 1, Luteovirus, Barley yellow dwarf virus, Machlomovirus, Maize chlorotic mottle virus, Marafivirus, Maize rayado fino virus, Microvirus, Coliphage phiX174, Spiromicrovirus, Spiroplasma phage 4, Bdellomicrovirus, Bdellovibrio phage MAC 1, Chlamydiamicrovirus, Chlamydia phage 1, T4-like phages, coliphage T4, Necrovirus, Tobacco necrosis virus, Nodavirus, Nodamura virus, Influenzavirus A, B and C, Thogoto virus, Polyomavirus, Murine polyomavirus, Papillomavirus, Rabbit (Shope) Papillomavirus, Paramyxovirus, Human parainfluenza virus 1, Morbillivirus, Measles virus, Rubulavirus, Mumps virus, Pneumovirus, Human respiratory syncytial virus, Partitivirus, Gaeumannomyces graminis virus 019/6-A, Chrysovirus, Penicillium chrysogenum virus, Alphacryptovirus, White clover cryptic viruses 1 and 2, Betacryptovirus, Parvovirinae, Parvovirus, Minute mice virus, Erythrovirus, B19 virus, Dependovirus, Adeno-associated virus 1, Densovirinae, Densovirus, Junonia coenia densovirus, Iteravirus, Bombyx mori virus, Contravirus, Aedes aegypti densovirus, Phycodnavirus, 1-Paramecium bursaria Chlorella NC64A virus group, Paramecium bursaria chlorella virus 1, 2-Paramecium bursaria Chlorella Pbi virus, 3-Hydra viridis Chlorella virus, Enterovirus, Human poliovirus 1. Rhinovirus Human rhinovirus 1A, Hepatovirus, Human hepatitis A virus, Cardiovirus, Encephalomyocarditis virus, Aphthovirus, Foot-and-mouth disease virus, Plasmavirus Acholeplasma phage L2, Podovirus, Coliphage T7, Ichnovirus, Campoletis sonorensis virus, Bracovirus, Cotesia melanoscela virus, Potexvirus, Potato virus X, Potyvirus, Potato virus Y, Rymovirus, Ryegrass mosaic virus, Bymovirus, Barley yellow mosaic virus, Orthopoxvirus, Vaccinia virus, Parapoxvirus, Orf virus, Avipoxvirus, Fowlpox virus, Capripoxvirus, Sheep pox virus, Leporipoxvirus, Myxoma virus, Suipoxvirus, Swinepox virus, Molluscipoxvirus, Molluscum contagiosum virus, Yatapoxvirus, Yaba monkey tumor virus, Entomopoxviruses A, B, and C, Melolontha melolontha entomopoxvirus, Amsacta moorei entomopoxvirus, Chironomus luridus entomopoxvirus, Orthoreovirus, Mammalian orthoreoviruses, reovirus 3, Avian orthoreoviruses, Orbivirus, African horse sickness viruses 1, Bluetongue viruses 1, Changuinola virus, Corriparta virus, Epizootic hemarrhogic disease virus 1, Equine encephalosis virus, Eubenangee virus group, Lebombo virus, Orungo virus, Palyam virus, Umatilla virus, Wallal virus, Warrego virus, Kemerovo virus, Rotavirus, Groups A-F rotaviruses, Simian rotavirus SA1l, Coltivirus, Colorado tick fever virus, Aquareovirus, Groups A-E aquareoviruses, Golden shiner virus, Cypovirus, Cypovirus types 1-12, Bombyx mori cypovirus 1, Fijivirus, Fijivirus groups 1-3, Fiji disease virus, Fijivirus groups 2-3, Phytoreovirus, Wound tumor virus, Oryzavirus, Rice ragged stunt, Mammalian type B retroviruses, Mouse mammary tumor virus, Mammalian type C retroviruses, Murine Leukemia Virus, Reptilian type C oncovirus, Viper retrovirus, Reticuloendotheliosis virus, Avian type C retroviruses, Avian leukosis virus, Type D Retroviruses, Mason-Pfizer monkey virus, BLV-HTLV retroviruses, Bovine leukemia virus, Lentivirus, Bovine lentivirus, Bovine immunodeficiency virus, Equine lentivirus, Equine infectious anemia virus, Feline lentivirus, Feline immunodeficiency virus, Canine immunodeficiency virus Ovine/caprine lentivirus, Caprine arthritis encephalitis virus, Visna/maedi virus, Primate lentivirus group, Human immunodeficiency virus 1, Human immunodeficiency virus 2, Human immunodeficiency virus 3, Simian immunodeficiency virus, Spumavirus, Human spuma virus, Vesiculovirus, Vesicular stomatitis Indiana virus, Lyssavirus, Rabies virus, Ephemerovirus, Bovine ephemeral fever virus, Cytorhabdovirus, Lettuce necrotic yellows virus, Nucleorhabdovirus, Potato yellow dwarf virus, Rhizidiovirus, Rhizidiomyces virus, Sequivirus, Parsnip yellow fleck virus, Waikavirus, Rice tungro spherical virus, Lambda-like phages, Coliphage lambda, Sobemovirus, Southern bean mosaic virus, Tectivirus, Enterobacteria phage PRDI, Tenuivirus, Rice stripe virus, Nudaurelia capensis beta-like viruses, Nudaurelia beta virus, Nudaurelia capensis omega-like viruses, Nudaurelia omega virus, Tobamovirus, Tobacco mosaic virus (vulgare strain: ssp. NC82 strain), Tobravirus, Tobacco rattle virus, Alphavirus, Sindbis virus, Rubivirus, Rubella virus, Tombusvirus, Tomato bushy stunt, virus, Carmovirus, Carnation mottle virus, Turnip crinkle virus, Totivirus, Saccharomyces cerevisiae virus, Giardiavirus, Giardia lamblia virus, Leishmaniavirus, Leishmania brasiliensis virus 1-1, Trichovirus, Apple chlorotic leaf spot virus, Tymovirus, Turnip yellow mosaic virus, Umbravirus, and Carrot mottle virus.

In some embodiments, the siRNA of the compositions described herein may inhibit a portion of the RNA genome of severe acute respiratory syndrome coronavirus 2 (SARS-CoV-2) (GenBank MN908947.3). In some embodiments, the siRNA of the compositions described herein may inhibit a portion of the RNA genome of SARS-COV-2 that encodes for the orflab protein (GenBank QHD43415.1), the SARS-COV-2 surface glycoprotein (GenBank QHD43416.1), the ORF3a protein (GenBank QHD43417.1), the SARS-COV-2 envelope protein (GenBank QHD43418.1), the SARS-COV-2 membrane glycoprotein (QHD43419.1), the ORF6 protein (GenBank QHD43420.1), the ORF7a protein (GenBank QHD43421.1), the ORF8 protein (GenBank QHD43422.1), the SARS-COV-2 nucleocapsid phosphoprotein (GenBank QHD43423.2), or the ORF10 protein (QHI42199.1).

The siRNA as used in the present invention may be modified, for example, to enhance efficacy and/or to reduce immune responsivity, by using, for example, base modifications or end-capping. In other embodiments, an unmodified siRNA is used in the present invention. Additionally, the siRNA as used in the present invention may comprise modified nucleic acid bases, such as modified DNA bases or modified RNA bases. Modifications may occur at, but are not restricted to, the sugar 2′ position, the C-5 position of pyrimidines, and the 8-position of purines.

Examples of suitable modified DNA or RNA bases include 2′-fluoro nucleotides, 2′-amino nucleotides, 5′-aminoallyl-2′-fluoro nucleotides and phosphorothioate nucleotides (monothiophosphate and dithiophosphate). Alternatively, the siRNA may comprise a nucleotide mimic. Examples of nucleotide mimics include locked nucleic acids (LNA), peptide nucleic acids (PNA), and phosphorodiamidate morpholino oligomers (PMO).

In some embodiments, the siRNA comprises at least one chemically modified nucleotide. In some embodiments, the at least one chemically modified nucleotide comprises a chemically modified nucleobase, a chemically modified ribose, a chemically modified phosphodiester linkage, or a combination thereof.

In some embodiments, the at least one chemically modified nucleotide is a chemically modified ribose. In some embodiments, the chemically modified ribose is 2′-O-methyl (2′-O-Me or 2′MeO or 2′-MeO) or 2′-fluoro (2′-F). In some embodiments, the chemically modified ribose is 2′-O-methyl (2′MeO). In some embodiments, the chemically modified ribose is 2′-fluoro (2′-F).

In some embodiments, the at least one chemically modified nucleotide is a chemically modified phosphodiester linkage. In some embodiments, the chemically modified phosphodiester linkage is phosphorothioate (PS). In some embodiments, all the nucleotides comprise chemically modified phosphodiester linkages.

### Peptide-siRNA Complex

In another aspect, a peptide and an siRNA of the invention associate to form a complex. As used herein, the term “associate” may refer to the interaction of a peptide and an siRNA through non-covalent bonds, or to the covalent bonding of a peptide and an siRNA. In some embodiments, a peptide and an siRNA of the invention associate through non-covalent bonds such as a hydrogen bond, an ionic bond, a bond based on Van Der Waals, a hydrophobic bond, or electrostatic interactions. For instance, a peptide of the invention may have an overall net positive charge, which may allow the peptide to associate with an siRNA of the invention through electrostatic interactions and/or hydrogen bonding to form a complex of the invention.

The molar ratio of peptide to siRNA at which a peptide of the invention associated with an siRNA of the invention can and will vary depending on the peptide, the siRNA composition, or the size of the siRNA, and may be determined experimentally. In essence, a suitable molar ration of a peptide of the invention to an siRNA of the invention may be a molar ratio wherein the peptide completely complexes the siRNA, while minimizing exposure of a subject to the peptide. In preferred embodiments of the present invention, the ratio is about 100:1 peptide:siRNA.

For instance, a peptide of the invention may associate with an siRNA in a peptide to siRNA molar ratio of about 50:1 to about 100:1. In some embodiments, the peptide to siRNA molar ratio may be about 50:1, about 55:1, about 60:1, about 65:1, about 70:1, about 75:1, about 80: 1, about 85:1, about 90: 1, about 95:1, or about 100:1. In some embodiments, the peptide to siRNA molar ratio may be about 50:1, 51:1, 52:1, 53:1, 54:1, 55:1, 56:1, 57:1, 58:1, or 59:1. In some embodiments, the peptide to siRNA ratio may be about 60:1, 61:1, 62:1, 63:1, 64:1, 65:1, 66:1, 67: 1, 68:1, or 69:1. In some embodiments, the peptide to siRNA ratio may be about 70:1, 71:1, 72:1, 73:1, 74:1, 75:1, 76:1, 77:1, 78:1, or 79:1. In some embodiments, the peptide to siRNA ratio may be about 80:1, 81:1, 82:1, 83:1, 84:1, 85:1, 86:1, 87:1, 88:1, or 89:1. In some embodiments, the peptide to siRNA ratio may be about 90:1, 91:1, 92: 1, 93:1, 94:1, 95:1, 96: 1, 97:1, 98:1, 99: 1, or 100:1.

Methods of determining a molar ratio wherein the peptide is capable of completely complexing a polynucleotide are known in the art, and may include gel retardation assays as previously described. Methods of determining a molar ratio wherein exposure of a subject to the peptide are minimized are known in the art, and may include cytotoxicity measurements using increasing doses of the peptide.

A peptide-siRNA complex of the invention may be about 10 nm to about 150 nm in average diameter, more preferably about 40 nm to about 80 nm in average diameter. As such, a peptide-siRNA complex of the invention may be referred to as a “nanoparticle”. In some embodiments, the peptide-siRNA complex of the invention may be about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, or about 150 nm in average diameter. In some embodiments, the peptide-siRNA complex of the invention may be about 10 nm, about 15 nm, about 20 nm, about 25 nm, about 30 nm, about 35 nm, about 40 nm, about 45 nm, or about 50 nm in average diameter. In some embodiments, the peptide-siRNA complex of the invention may be about 55 nm, about 60 nm, about 65 nm, about 70 nm, about 75 nm, about 80 nm, about 85 nm, about 90 nm, about 95 nm, or about 100 nm in average diameter. In some embodiments, the peptide-siRNA complex of the invention may be about 105 nm, about 110 nm, about 115 nm, about 120 nm, about 125 nm, about 130 nm, about 135 nm, about 140 nm, about 145 nm, or about 150 nm in average diameter. In some embodiments, the peptide-siRNA complex of the invention may be about 40 nm, about 42 nm, about 44 nm, about 46 nm, about 48 nm, about 50 nm, about 52 nm, about 54 nm, about 56 nm, about 58 nm, about 60 nm, about 62 nm, about 64 nm, about 66 nm, about 68 nm, about 70 nm, about 72 nm, about 74 nm, about 76 nm, about 78 nm, or about 80 nm in average diameter.

In some embodiments, a nanoparticle comprising a peptide-siRNA complex of the invention may comprise an aggregate of smaller particles of about 5 to about 20 nm in average diameter. As such, a nanoparticle of the invention may comprise an aggregate of smaller particles of about 5, 6, 7, 8, 9, 10, 11, 12, 13, 14, 15, 16, 17, 18, 19, or 20 nm in average diameter.

Particle size may be assessed using methods known in the art. Non-limiting examples of methods of measuring the size of a particle may include dynamic light scattering, atomic force microscopy, scanning electron microscopy, transmission electron microscopy, laser diffraction, electrozone (electric sensing zone), light obscuration (also referred to as photozone and single particle optical sensing or SPOS), sieve analysis, aerodynamic measurements, air permeability diameter, sedimentation, or combinations thereof. In some embodiments, particle size is assessed by dynamic light scattering or by atomic force microscopy.

A nanoparticle of the invention may have a zeta potential of about −15 to about 20 mV, preferably about 0 mV or more. For instance, a nanoparticle may have a zeta potential of about 1, 2, 3, 4, 5, 6, 7, 8, 9, 10, 11, 12, 13, 14, 15, 16, 18, 18, 19, or 20 mV or more. In some embodiments, a nanoparticle has a zeta potential of about 1, about 2, about 3, about 4, or about 5 mV. In other embodiments, a nanoparticle has a zeta potential about 10, about 11, about 12, about 13, or about 14 mV. In other embodiments, a nanoparticle has a zeta potential of about 11, about 12, about 13, about 14, or about 15 mV.

A nanoparticle comprising a peptide-siRNA complex of the invention may have a positive to negative charge ratio of about 1:1 to about 30:1, preferably about 10:1 to about 25:1. In some embodiments, a nanoparticle has a positive to negative charge ratio of about 4:1, about 5:1, about 6: 1, about 7:1, or about 8:1. In other embodiments, a nanoparticle has a positive to negative charge ratio of about 10:1, about 11:1, about 12:1, about 13:1, or about 14:1. In other embodiments, a nanoparticle has a positive to negative charge ratio of about 22:1, about 23:1, about 24:1, about 25:1, or about 26:1.

In some embodiments, the peptide-siRNA complex comprises a ratio of peptide:siRNA that is more than about 50:1 and less than about 200:1. In some embodiments, the peptide-siRNA complex comprises a ratio of peptide:siRNA that is less than about 50:1. The molar ratio of the peptide to siRNA at which the peptide associates with a polynucleotide of the invention can and will vary depending on the peptide, the siRNA composition, or the size of the siRNA, and may be determined by one of skill in the art. In essence, a suitable molar ratio of a peptide of the invention to a siRNA of the invention may be a molar ratio wherein the peptide completely complexes the siRNA. For instance, a peptide may associate with a siRNA in a peptide to siRNA molar ratio of about 1:1, 10:1, 20:1, 30:1, 40:1, 50:1, 60: 1, 70:1, 80:1, 90:1, 100:1, 110:1, 120:1, 130:1, 140:1, 150:1, 160:1, 170:1, 180:1, 190:1, 200:1, 210: 1, 220:1, 230:1, 240:1, 250:1, 260: 1, 270:1, 280:1, 290:1, or about 300:1 or more. In some embodiments, a peptide may associate with a siRNA in a peptide to siRNA molar ratio of about 20:1 to about 250:1. In some embodiments, a peptide may associate with a siRNA in a peptide to siRNA molar ratio of about 50:1 to about 200:1. In some embodiments, a peptide may associate with a siRNA in a peptide to siRNA molar ratio of about 75:1 to about 225:1. In some embodiments, a peptide may associate with a siRNA in a peptide to siRNA molar ratio of about 100:1 to about 200:1. In some embodiments, a peptide may associate with a siRNA in a peptide to siRNA molar ratio of about 125:1 to about 175:1.

Depending on the size and charge of the siRNA, the molar ratios can be larger. Thus, in other embodiments, a peptide may associate with a siRNA in a peptide to siRNA molar ratio of about 300:1, 400:1, 500:1, 600:1, 700:1, 800:1, 900:1, 1000:1, 1500:1, 2000:1, 2500:1, 3000:1, or more. In some embodiments, the peptide-siRNA complex comprises a ratio of peptide:siRNA that is more than about 300:1 and less than about 1000:1. In some embodiments, the peptide-siRNA complex comprises a ratio of peptide:siRNA that is more than about 1000:1 and less than about 3000:1.

A peptide-siRNA complex is capable of efficient release of the siRNA into the cytoplasm of a virus-infected cell. The peptide-siRNA complex may also be capable of protecting the siRNA from degradation upon administration in a subject. As such, a peptide-siRNA nanoparticle of the invention may remain stable in the presence of serum. A nanoparticle may remain stable in the presence of serum for about 10, 20, 30, 40, 50, 60 minutes, about 1, 2, 3, 4, 5, 6, 7, 8, 9, 10, 11, 12, 13, 14, 15, 16, 17, 18, 19 hours, about 1, 2, 3, 4, 5, 6, 7 days or longer. Stability of the nanoparticle may be determined by measuring the ability of a nanoparticle to maintain the activity of an siRNA of the peptide-siRNA complex of the nanoparticle, or by measuring changes in the size of the nanoparticle over time. Methods of measuring the size of a nanoparticle may be as described herein.

Methods of preparing a peptide-siRNA complex of the invention generally comprise contacting a peptide of the invention with an siRNA of the invention to form a peptide-siRNA complex. Typically, a peptide and an siRNA are contacted by incubating under conditions suitable for a peptide-siRNA complex to from. Typically, such conditions may comprise a temperature of about 30° C. to about 40° C., and incubation times of between about 20 see to about 60 min or more. Suitable temperatures may also be lower than about 30° C. For example, incubation may occur on ice. One skilled in the art will appreciate that the length and temperature of incubation can and will vary depending on the peptide and the siRNA, and may be determined experimentally.

In another aspect, the peptide-siRNA complex of the present invention comprises a hyaluronic acid. In some embodiments, the hyaluronic acid coats the peptide-siRNA complex. In some embodiments, the hyaluronic acid is integrated into the peptide-siRNA complex. The hyaluronic may have an average molecular weight ranging from about 5 to about 20,000 kDa, for example from about 3,000 to about 4,000 kDa.

In some embodiments, the hyaluronic acid comprises a hyaluronic acid conjugate. In some embodiments, the hyaluronic acid conjugate comprises a hyaluronic acid covalently bound to a targeting ligand that directs the peptide-siRNA complex to the desired infected cell type. The hyaluronic acid may be directly covalently bound to the targeting ligand or may be bound through the use of a covalent linker group.

In one non-limiting representative embodiment, the hyaluronic acid conjugate comprises hyaluronic acid covalently bound to an angiotensin-converting enzyme 2 (ACE2) ligand. The ACE2 ligand can be, for example, a protein ligand, a small organic molecule ligand, or an aptamer ligand. Representative examples of ACE2 ligands are known within the art.

A peptide-siRNA complex may be incubated with a hyaluronic acid for about 5, 10, 15, 20, 25, 30, 35, 40, 45, 50, 55, or about 60 minutes or more to allow the hyaluronic acid to coat the peptide-siRNA complex or to integrate into the peptide-siRNA complex. A peptide-siRNA complex may be incubated with a hyaluronic acid for about 1, 2, 3, 4, 5, 10, 12, 18, or 24 hours or more, to allow the hyaluronic acid to coat the peptide-siRNA complex or to integrate into the peptide-siRNA complex. In some embodiments, a peptide-siRNA complex may be incubated with a hyaluronic acid for about 45 minutes. Shorter times could be used in some embodiments, for example, when using microfluidic devices.

In another aspect, the present disclosure provides a composition comprising a peptide-siRNA complex, wherein the peptide-siRNA complex comprises: a peptide: a small interfering RNA (siRNA) that interferes with a ribonucleotide in a cell that encodes a protein that promotes assembly of a virus; and a hyaluronic acid (HA): wherein the peptide is non-lytic and capable of affecting release of the siRNA from an endosome of a cell; and wherein the peptide comprises an amino acid sequence with at least 80% identity to the amino acid sequence of SEQ ID NO: 1.

In yet another aspect, the present disclosure provides a composition comprising a peptide-siRNA complex, wherein the peptide-siRNA complex comprises: a peptide: a first small interfering RNA (siRNA) that interferes with a viral ribonucleotide: a second small interfering RNA (siRNA) that interferes with a ribonucleotide in a cell that encodes a protein that promotes assembly of a virus; and a hyaluronic acid (HA): wherein the peptide is non-lytic and capable of affecting release of the siRNA from an endosome of a virus-infected cell; and wherein the peptide comprises an amino acid sequence with at least 80% identity to the amino acid sequence of SEQ ID NO: 1.

### Pharmaceutical Compositions

In another aspect, a composition described herein may comprise an excipient. This composition may be useful in a variety of medical and non-medical applications. For example, pharmaceutical compositions may be useful in the delivery of an effective amount of a polynucleotide to a subject in need thereof.

In certain embodiments, the polynucleotide is DNA or RNA. In certain embodiments, the RNA is RNAi, dsRNA, siRNA, shRNA, miRNA, or antisense RNA. In certain embodiments, the compositions comprise one or more additional active compounds. In certain embodiments, the composition further comprises an agent. For example, in certain embodiments, the agent is a small molecule, organometallic compound, nucleic acid, protein, peptide, polynucleotide, metal, targeting agent, an isotopically labeled chemical compound, drug, vaccine, immunological agent, or an agent useful in bioprocessing.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy. 21st Edition (Lippincott Williams & Wilkins, 2005).

Exemplary excipients include, but are not limited to, any non-toxic, inert solid, semi-solid or liquid filler, diluent, encapsulating material or formulation auxiliary of any type. Some examples of materials which can serve as excipients include, but are not limited to, sugars such as lactose, glucose, and sucrose: starches such as corn starch and potato starch: cellulose and its derivatives such as sodium carboxymethyl cellulose, ethyl cellulose, and cellulose acetate; powdered tragacanth; malt: gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxy benzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxy benzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers rs such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxy methylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, and fibrin, other polymers, for example, polyhydroxyacids such as polylactide, polyglycolide, polyl(lactide-co-glycolide) and poly(·epsilon·-caprolactone-co-glycolide)-, carboxyvinyl polymers and their salts (e.g., carbomer), polyvinylpyrrolidone (PVP), polyacrylic acid and its salts, polyacrylamide, polyacilic acid/acrylamide copolymer, polyalkylene oxides such as polyethylene oxide, polypropylene oxide, poly(ethylene oxide-propylene oxide), and a Pluronic polymer, polyoxyethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy (Polyethylene glycol)-1000], 1,2-Distearoyl-sn-glycero-3-Phosphoethanolamine-N-[Methoxy (Polyethylene glycol)-2000], and 1,2-Distearoyl-sn-glycero-3-Phosphoethanolamine-N-[Methoxy (Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxyalkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) from the composition only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The composition is admixed with an excipient and any needed preservatives or buffers as may be required. Ophthalmic formulation, ear drops, and eye drops are also contemplated as being within the scope of this invention.

The ointments, pastes, creams, and gels may contain excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a composition to the body. Such dosage forms can be made by dissolving or dispensing the compositions in a proper medium. Absorption enhancers can also be used to increase the flux of the compound across the skin. The rate can be controlled by either providing a rate controlling membrane or by dispersing the particles in a polymer matrix or gel.

### Methods of Treatment

The present disclosure also provides methods for treating a viral infection in a subject in need thereof comprising administering a therapeutically effective amount of a composition as described herein.

Viruses that may be treated by the methods described herein can include both DNA viruses and RNA viruses. Exemplary viruses can belong to the following none exclusive list of families: Adenoviridae, Arenaviridae, Astroviridae, Baculoviridae, Barnaviridae, Betaherpesvirinae, Birnaviridae, Bromoviridae, Bunyaviridae, Caliciviridae, Chordopoxvirinae, Circoviridae, Comoviridae, Coronaviridae, Cystoviridae, Corticoviridae, Entomopoxvirinae, Filoviridae, Flaviviridae, Fuselloviridae, Geminiviridae, Hepadnaviridae, Herpesviridae, Gammaherpesvirinae, Inoviridae, Iridoviridae, Leviviridae, Lipothrixviridae, Microviridae, Myoviridae, Nodaviridae, Orthomyxoviridae, Papovaviridae, Paramyxoviridae, Paramyxovirinae, Partitiviridae, Parvoviridae, Phycodnaviridae, Picornaviridae, Plasmaviridae, Pneumovirinae, Podoviridae, Polydnaviridae, Potyviridae, Poxviridae, Reoviridae, Retroviridae, Rhabdoviridae, Sequiviridae, Siphoviridae, Tectiviridae, Tetraviridae, Togaviridae, Tombusviridae, and Totiviridae.

Specific examples of suitable viruses include, but are not limited to, Mastadenovirus, Human adenovirus 2, Aviadenovirus, African swine fever virus, arenavirus, Lymphocytic choriomeningitis virus, Ippy virus, Lassa virus, Arterivirus, Human astrovirus 1, Nucleopolyhedrovirus, Autographa californica nucleopolyhedrovirus, Granulovirus, Plodia interpunctella granulovirus, Badnavirus, Commelina yellow mottle virus, Rice tungro bacilliform, Barnavirus, Mushroom bacilliform virus, Aquabirnavirus, Infectious pancreatic necrosis virus, Avibirnavirus, Infectious bursal disease virus, Entomobirnavirus, Drosophila X virus, Alfamovirus, Alfalfa mosaic virus, Ilarvirus, Ilarvirus Subgroups 1-10, Tobacco streak virus, Bromovirus, Brome mosaic virus, Cucumovirus, Cucumber mosaic virus, Bhanja virus Group, Kaisodi virus, Mapputta virus, Okola virus, Resistencia virus, Upolu virus, Yogue virus, Bunyavirus, Anopheles A virus, Anopheles B virus, Bakau virus, Bunyamwera virus, Bwamba virus, C virus, California encephalitis virus, Capim virus, Gamboa virus, Guama virus, Koongol virus, Minatitlan virus, Nyando virus, Olifantsvlei virus, Patois virus, Simbu virus, Tete virus, Turlock virus, Hantavirus, Hantaan virus, Nairovirus, Crimean-Congo hemorrhagic fever virus, Dera Ghazi Khan virus, Hughes virus, Nairobi sheep disease virus, Qalyub virus, Sakhalin virus, Thiafora virus, Crimean-congo hemorrhagic fever virus, Phlebovirus, Sandfly fever virus, Bujaru complex, Candiru complex, Chilibre complex, Frijoles complex, Punta Toro complex, Rift Valley fever complex, Salehabad complex, Sandfly fever Sicilian virus, Uukuniemi virus, Uukuniemi virus, Tospovirus, Tomato spotted wilt virus, Calicivirus, Vesicular exanthema of swine virus, Capillovirus, Apple stem grooving virus, Carlavirus, Carnation latent virus, Caulimovirus, Cauliflower mosaic virus, Circovirus, Chicken anemia virus, Closterovirus, Beet yellows virus, Comovirus, Cowpea mosaic virus, Fabavirus, Broad bean wilt virus 1, Nepovirus, Tobacco ringspot virus, Coronavirus, Avian infectious bronchitis virus, Bovine coronavirus, Canine coronavirus, Feline infectious peritonitis virus, Human coronavirus 299E, Human coronavirus OC43, Murine hepatitis virus, Porcine epidemic diarrhea virus, Porcine hemagglutinating encephalomyelitis virus, Porcine transmissible gastroenteritis virus, Rat coronavirus, Turkey coronavirus, Rabbit coronavirus, Torovirus, Berne virus, Breda virus, Corticovirus, Alteromonas phage PM2, Pseudomonas Phage phi6, Deltavirus, Hepatitis delta virus, Dianthovirus Carnation ringspot virus, Red clover necrotic mosaic virus, Sweet clover necrotic mosaic virus, Enamovirus, Pea enation mosaic virus, Filovirus, Marburg virus, Ebola virus Zaire, Flavivirus, Yellow fever virus, Tick-borne encephalitis virus, Rio Bravo Group, Japanese encephalitis, Tyuleniy Group, Ntaya Group, Uganda S Group, Dengue Group, Modoc Group, Pestivirus, Bovine diarrhea virus, Hepatitis C virus, Furovirus, Soil-borne wheat mosaic virus, Beet necrotic yellow vein virus, Fusellovirus, Sulfobolus virus 1, Subgroup I, II, and III geminivirus, Maize streak virus, Beet curly top virus, Bean golden mosaic virus, Orthohepadnavirus, Hepatitis B virus, Avihepadnavirus, Alphaherpesvirinae, Simplexvirus, Human herpesvirus 1, Varicellovirus, Human herpesvirus 3, Cytomegalovirus, Human herpesvirus 5. Muromegalovirus, Mouse cytomegalovirus 1, Roseolovirus, Human herpesvirus 6, Lymphocryptovirus, Human herpesvirus 4, Rhadinovirus, Ateline herpesvirus 2, Hordeivirus, Barley stripe mosaic virus, Hypoviridae, Hypovirus, Cryphonectria hypovirus 1-EP713, Idaeovirus, Raspberry bushy dwarf virus, Inovirus, Coliphage fd, Plectrovirus, Acholeplasma phage L51, Iridovirus, Chilo iridescent virus, Chloriridovirus, Mosquito iridescent virus, Ranavirus, Frog virus 3, Lymphocystivirus, Lymphocystis disease virus flounder isolate, Goldfish virus 1, Levivirus, Enterobacteria phage MS2, Allolevirus, Enterobacteria phage Qbeta, Lipothrixvirus, Thermoproteus virus 1, Luteovirus, Barley yellow dwarf virus, Machlomovirus, Maize chlorotic mottle virus, Marafivirus, Maize rayado fino virus, Microvirus, Coliphage phiX174, Spiromicrovirus, Spiroplasma phage 4, Bdellomicrovirus, Bdellovibrio phage MAC 1, Chlamydiamicrovirus, Chlamydia phage 1, T4-like phages, coliphage T4, Necrovirus, Tobacco necrosis virus, Nodavirus, Nodamura virus, Influenzavirus A, B and C, Thogoto virus, Polyomavirus, Murine polyomavirus, Papillomavirus, Rabbit (Shope) Papillomavirus, Paramyxovirus, Human parainfluenza virus 1. Morbillivirus, Measles virus, Rubulavirus, Mumps virus, Pneumovirus, Human respiratory syncytial virus, Partitivirus, Gaeumannomyces graminis virus 019/6-A, Chrysovirus, Penicillium chrysogenum virus, Alphacryptovirus, White clover cryptic viruses 1 and 2, Betacryptovirus, Parvovirinae, Parvovirus, Minute mice virus, Erythrovirus, B19 virus, Dependovirus, Adeno-associated virus 1, Densovirinae, Densovirus, Junonia coenia densovirus, Iteravirus, Bombyx mori virus, Contravirus, Aedes aegypti densovirus, Phycodnavirus, 1-Paramecium bursaria Chlorella NC64A virus group, Paramecium bursaria chlorella virus 1, 2-Paramecium bursaria Chlorella Pbi virus, 3-Hydra viridis Chlorella virus, Enterovirus, Human poliovirus 1, Rhinovirus Human rhinovirus 1A, Hepatovirus, Human hepatitis A virus, Cardiovirus, Encephalomyocarditis virus, Aphthovirus, Foot-and-mouth disease virus, Plasmavirus Acholeplasma phage L2, Podovirus, Coliphage T7, Ichnovirus, Campoletis sonorensis virus, Bracovirus, Cotesia melanoscela virus, Potexvirus, Potato virus X, Potyvirus, Potato virus Y, Rymovirus, Ryegrass mosaic virus, Bymovirus, Barley yellow mosaic virus, Orthopoxvirus, Vaccinia virus, Parapoxvirus, Orf virus, Avipoxvirus, Fowlpox virus, Capripoxvirus, Sheep pox virus, Leporipoxvirus, Myxoma virus, Suipoxvirus, Swinepox virus, Molluscipoxvirus, Molluscum contagiosum virus, Yatapoxvirus, Yaba monkey tumor virus, Entomopoxviruses A, B, and C, Melolontha melolontha entomopoxvirus, Amsacta moorei entomopoxvirus, Chironomus luridus entomopoxvirus, Orthoreovirus, Mammalian orthoreoviruses, reovirus 3, Avian orthoreoviruses, Orbivirus, African horse sickness viruses 1, Bluetongue viruses 1, Changuinola virus, Corriparta virus, Epizootic hemarrhogic disease virus 1, Equine encephalosis virus, Eubenangee virus group, Lebombo virus, Orungo virus, Palyam virus, Umatilla virus, Wallal virus, Warrego virus, Kemerovo virus, Rotavirus, Groups A-F rotaviruses, Simian rotavirus SA1l, Coltivirus, Colorado tick fever virus, Aquareovirus, Groups A-E aquareoviruses, Golden shiner virus, Cypovirus, Cypovirus types 1-12, Bombyx mori cypovirus 1, Fijivirus, Fijivirus groups 1-3, Fiji disease virus, Fijivirus groups 2-3, Phytoreovirus, Wound tumor virus, Oryzavirus, Rice ragged stunt, Mammalian type B retroviruses, Mouse mammary tumor virus, Mammalian type C retroviruses, Murine Leukemia Virus, Reptilian type C oncovirus, Viper retrovirus, Reticuloendotheliosis virus, Avian type C retroviruses, Avian leukosis virus, Type D Retroviruses, Mason-Pfizer monkey virus, BLV-HTLV retroviruses, Bovine leukemia virus, Lentivirus, Bovine lentivirus, Bovine immunodeficiency virus, Equine lentivirus, Equine infectious anemia virus, Feline lentivirus, Feline immunodeficiency virus, Canine immunodeficiency virus Ovine/caprine lentivirus, Caprine arthritis encephalitis virus, Visna/maedi virus, Primate lentivirus group, Human immunodeficiency virus 1, Human immunodeficiency virus 2, Human immunodeficiency virus 3, Simian immunodeficiency virus, Spumavirus, Human spuma virus, Vesiculovirus, Vesicular stomatitis Indiana virus, Lyssavirus, Rabies virus, Ephemerovirus, Bovine ephemeral fever virus, Cytorhabdovirus, Lettuce necrotic yellows virus, Nucleorhabdovirus, Potato yellow dwarf virus, Rhizidiovirus, Rhizidiomyces virus, Sequivirus, Parsnip yellow fleck virus, Waikavirus, Rice tungro spherical virus, Lambda-like phages, Coliphage lambda, Sobemovirus, Southern bean mosaic virus, Tectivirus, Enterobacteria phage PRDI, Tenuivirus, Rice stripe virus, Nudaurelia capensis beta-like viruses, Nudaurelia beta virus, Nudaurelia capensis omega-like viruses, Nudaurelia omega virus, Tobamovirus, Tobacco mosaic virus (vulgare strain: ssp. NC82 strain), Tobravirus, Tobacco rattle virus, Alphavirus, Sindbis virus, Rubivirus, Rubella virus, Tombusvirus, Tomato bushy stunt, virus, Carmovirus, Carnation mottle virus, Turnip crinkle virus, Totivirus, Saccharomyces cerevisiae virus, Giardiavirus, Giardia lamblia virus, Leishmaniavirus, Leishmania brasiliensis virus 1-1, Trichovirus, Apple chlorotic leaf spot virus, Tymovirus, Turnip yellow mosaic virus, Umbravirus, and Carrot mottle virus.

In some embodiments, the compositions described herein may be used in the treatment of an infection caused by the SARS-COV-2 virus.

The composition may be administered in such amounts, time, and route deemed necessary in order to achieve the desired result. The exact amount of the composition will vary from subject to subject, depending on the species, age, and general condition of the subject, the severity of the infection, the particular composition, its mode of administration, its mode of activity, and the like. The composition is preferably formulated in dosage unit form for ease of administration and uniformity of dosage. It will be understood, however, that the total daily usage of the composition will be decided by the attending physician within the scope of sound medical judgment. The specific therapeutically effective dose level for any particular subject will depend upon a variety of factors including the disorder being treated and the severity of the disorder: the activity of the composition employed: the specific composition employed: the age, body weight, general health, sex and diet of the patient: the time of administration, route of administration, and rate of excretion of the specific composition employed: the duration of the treatment: drugs used in combination or coincidental with the specific composition employed; and like factors well known in the medical arts.

The composition may be administered by any route. In some embodiments, the composition is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual: by intratracheal instillation, bronchial instillation, and/or inhalation; and/or as an oral spray, nasal spray, and/or aerosol. In general, the most appropriate route of administration will depend upon a variety of factors including the nature of the composition (e.g., its stability in the environment of the gastrointestinal tract), the condition of the subject (e.g., whether the subject is able to tolerate oral administration), etc.

The exact amount of a composition required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

### Kits

Another aspect of the invention encompasses a kit for preparing a peptide-siRNA complex. In some embodiments, the kit comprises a first composition comprising a peptide, a second composition comprising an siRNA, and a third composition comprising a hyaluronic acid (HA). By following directions provided by the kit, a user of the kit may mix the composition comprising a peptide and the composition comprising an siRNA to form a peptide-siRNA complex, followed by incubation with a hyaluronic acid. The directions of the kit may include instructions to mix the peptide and siRNA at a suitable ratio. Suitable ratios are described above. The kit may also include suitable buffers, water, or cross-linking reagents.

Further embodiments of the present disclosure are provided below:

Embodiment 1: A composition comprising a peptide-siRNA complex, wherein the peptide-siRNA complex comprises:
a peptide,
a small interfering RNA (siRNA) that interferes with a viral ribonucleotide, and
a hyaluronic acid;

wherein the peptide is non-lytic in circulation and capable of affecting release of the siRNA from an endosome in a virus-infected cell; and
wherein the peptide comprises an amino acid sequence with at least 80% identity to the amino acid sequence of SEQ ID NO: 1.

Embodiment 2: The composition of embodiment 1, wherein the peptide-siRNA complex is about 10 nm to about 150 nm in average diameter.

Embodiment 3: The composition of any one of embodiments 1 or 2, wherein the peptide-siRNA complex is about 40 nm to about 80 nm in average diameter.

Embodiment 4: The composition of any one of embodiments 1-3, wherein the hyaluronic acid coats the peptide-siRNA complex.

Embodiment 5: The composition of any one of embodiments 1-3, wherein the hyaluronic acid is integrated into the peptide-siRNA complex.

Embodiment 6: The composition of any one of embodiments 1-5, wherein the hyaluronic acid comprises a hyaluronic acid conjugate.

Embodiment 7: The composition of embodiment 6, wherein the hyaluronic acid conjugate comprises hyaluronic acid covalently bound to a cell-targeting ligand.

Embodiment 8: The composition of any one of embodiments 6 or 7, wherein the hyaluronic acid conjugate comprises hyaluronic acid covalently bound to an angiotensin-converting enzyme 2 (ACE2) ligand.

Embodiment 9: The composition of any one of embodiments 1-8, wherein the peptide comprises an amino acid sequence with at least 85% identity to the amino acid sequence of SEQ ID NO: 1.

Embodiment 10: The composition of embodiment 9, wherein the peptide comprises an amino acid sequence with at least 90% identity to the amino acid sequence of SEQ ID NO: 1.

Embodiment 11: The composition of embodiment 10, wherein the peptide comprises an amino acid sequence with at least 95% identity to the amino acid sequence of SEQ ID NO: 1.

Embodiment 12: The composition of embodiment 11, wherein the peptide comprises SEQ ID NO: 1.

Embodiment 13: The composition of any one of embodiments 1-8, wherein the peptide consists of an amino acid sequence with at least 85% identity to the amino acid sequence of SEQ ID NO: 1.

Embodiment 14: The composition of embodiment 13, wherein the peptide consists of an amino acid sequence with at least 90% identity to the amino acid sequence of SEQ ID NO: 1.

Embodiment 15: The composition of embodiment 14, wherein the peptide consists of an amino acid sequence with at least 95% identity to the amino acid sequence of SEQ ID NO: 1.

Embodiment 16: The composition of embodiment 15, wherein the peptide consists of the amino acid sequence of SEQ ID NO: 1.

Embodiment 17: The composition of any one of embodiments 1-16, wherein the siRNA interferes with at least a portion of the RNA genome of severe acute respiratory syndrome coronavirus 2 (SARS-COV-2).

Embodiment 18: The composition of any one of embodiments 1-17, wherein the peptide-siRNA complex comprises a ratio of peptide:siRNA from about 50:1 to about 200:1.

Embodiment 19: A method of treating a viral infection in a subject in need thereof comprising administering to the subject a therapeutically effective amount of the composition of any one of embodiments 1-18.

Embodiment 20: The method of embodiment 19, wherein the subject is a human.

Embodiment 21: The method of any one of embodiments 19 or 20, wherein the viral infection comprises SARS-COV-2.

Embodiment 22: A method of delivering siRNA to a virus-infected cell comprising contacting the cell with the composition of any one of embodiments 1-18.

Embodiment 23: The method of embodiment 22, wherein the cell is infected with SARS-COV-2.

Embodiment 24: A kit for preparing a peptide-siRNA complex of any one of embodiments 1-18, the kit comprising:
a first composition comprising the peptide;
a second composition comprising the siRNA; and
a third composition comprising a hyaluronic acid (HA).

A number of embodiments of the disclosure have been described. Nevertheless, it will be understood that various modifications may be made without departing from the spirit and scope of the invention. Accordingly, other embodiments are within the scope of the following claims.

1. A composition comprising a peptide-siRNA complex, wherein the peptide-siRNA complex comprises:
a peptide,
a small interfering RNA (siRNA) that interferes with a viral ribonucleotide, and
a hyaluronic acid;
wherein the peptide is non-lytic in circulation and capable of affecting release of the siRNA from an endosome in a virus-infected cell; and
wherein the peptide comprises an amino acid sequence with at least 80% identity to the amino acid sequence of SEQ ID NO: 1.

2. The composition of claim 1, wherein the peptide-siRNA complex is about 10 nm to about 150 nm in average diameter.

3. The composition of claim 1, wherein the peptide-siRNA complex is about 40 nm to about 80 nm in average diameter.

4. The composition of claim 1, wherein the hyaluronic acid coats the peptide-siRNA complex.

5. The composition of claim 1, wherein the hyaluronic acid is integrated into the peptide-siRNA complex.

6. The composition of claim 1, wherein the hyaluronic acid comprises a hyaluronic acid conjugate.

7. The composition of claim 6, wherein the hyaluronic acid conjugate comprises hyaluronic acid covalently bound to a cell-targeting ligand.

8. The composition of claim 6, wherein the hyaluronic acid conjugate comprises hyaluronic acid covalently bound to an angiotensin-converting enzyme 2 (ACE2) ligand.

9. The composition of claim 1, wherein the peptide comprises an amino acid sequence with at least 85% identity, at least 90% identity, or at least 95% identity to the amino acid sequence of SEQ ID NO: 1.

10-11. (canceled)

12. The composition of claim 1, wherein the peptide comprises SEQ ID NO: 1.

13. The composition of claim 1, wherein the peptide consists of an amino acid sequence with at least 85% identity, at least 90% identity, or at least 95% identity to the amino acid sequence of SEQ ID NO: 1.

14-15. (canceled)

16. The composition of claim 1, wherein the peptide consists of the amino acid sequence of SEQ ID NO: 1.

17. The composition of claim 1, wherein the siRNA interferes with at least a portion of the RNA genome of severe acute respiratory syndrome coronavirus 2 (SARS-COV-2).

18. The composition of claim 1, wherein the peptide-siRNA complex comprises a ratio of peptide:siRNA from about 50:1 to about 200:1.

19. A method of treating a viral infection in a subject in need thereof comprising administering to the subject a therapeutically effective amount of the composition of of claim 1.

20. The method of claim 19, wherein the subject is a human.

21. The method of claim 19, wherein the viral infection comprises SARS-COV-2.

22. A method of delivering siRNA to a virus-infected cell comprising contacting the cell with the composition of claim 1.

23. The method of claim 22, wherein the cell is infected with SARS-COV-2.

24. A kit for preparing a peptide-siRNA complex of claim 1, the kit comprising:
a first composition comprising the peptide;
a second composition comprising the siRNA; and
a third composition comprising a hyaluronic acid (HA).


SEQUENCE LISTING (1 sequence)

SEQ ID NO: 1 - protein, 21 aa, Artificial Sequence
  complexing peptide
VLTTGLPALISWIRRRHRRHC